(19) United States
(12) Patent Application Publication
(10) Pub. No.: US 2018/0263560 A1
(43) Pub. Date: Sep. 20, 2018

(54) SYSTEM, DEVICE AND METHOD FOR MONITORING PHYSICAL RECOVERY

(71) Applicant: KINERIS INC., Kitchener (CA)

(72) Inventors: Ryan M. Bakker, Guelph (CA); Stefan S. TOMESCU, Toronto (CA); Gajendra A.R. HANGALUR, Kitchener (CA)

(21) Appl. No.: 15/984,024

(22) Filed: May 18, 2018

Related U.S. Application Data
(63) Continuation of application No. PCT/CA2016/051367, filed on Nov. 11, 2016.
(60) Provisional application No. 62/259,583, filed on Nov. 24, 2015.

(51) Int. Cl. A61B 5/00; A61B 5/0492; A61B 5/11

(52) U.S. Cl. CPC A61B 5/4884; A61B 5/4824; A61B 5/0492; A61B 5/1123; A61B 2562/0215; A61B 5/6804; A61B 5/4585; A61B 2562/0219; A61B 2505/09; A61B 5/1121

(57) ABSTRACT

Described are various embodiments of a system, device and method for monitoring physical recovery, for instance, that allow for the effective monitoring of pain, and the evolution thereof, during such recovery, in some examples, within the context of a rehabilitation or recovery training program that may involve a series of prescribed exercise routines and the like.

### CROSS REFERENCE TO RELATED APPLICATIONS

This application is a continuation of International Patent Application No. PCT/CA2016/051367 entitled “SYSTEM, DEVICES AND METHOD FOR MONITORING PHYSICAL RECOVERY”, filed Nov. 22, 2016, which claims priority to U.S. Provisional Patent Application No. 62/259,583 entitled “METHOD AND SYSTEM FOR THE MONITORING AND ANALYSIS OF JOINT INJURIES AND DISEASE”, filed Nov. 24, 2015, each of which is incorporated by reference in its entirety.

### FIELD OF THE DISCLOSURE

The present disclosure relates to physical treatment, and, in particular, to a system, device and method for monitoring physical recovery.

### BACKGROUND

Many people suffer from joint, muscular and/or other physical injuries and diseases. Physical injuries are induced from a number of causes. Examples include traumatic accidents such as car crashes and sporting accidents. Diseases such as osteoarthritis can also affect normal function. Upon seeking treatment for these injuries and disease, patients often will meet with multiple healthcare professionals such as a primary care physician, surgeon or physiotherapist. These professionals will instruct the patient how to best care for their injury or disease.

Physiotherapy and exercise are the primary recommendations for the rehabilitation and management of muscle and joint injuries. Patients who undergo physiotherapy and exercise for rehabilitation often see faster recovery rates, increased range of motion and improved muscle function. Patients who do not perform physiotherapy often see a delayed or incomplete recovery.

During physiotherapy a patient will typically receive a set of prescribed exercises and stretches to be performed both at the physiotherapy clinic and at home. These exercises are often accompanied by a document, which gives the patient instructions of each exercise, how many days to complete the exercise, number of exercise sets and number of repetitions. The exercises are determined by the physiotherapist to guide the patient through the rehabilitation protocol to increase speed and quality of the recovery. The patient adherence to the protocol is important to receive the full benefits of physiotherapy. Rehabilitative therapy is often concluded when the patient has met their goals for recovery. These goals will often relate to pain and a return to normal function.

The primary metrics used to assess the rehabilitation of an injury by healthcare professionals, are the return of range of motion, increases in muscle strength and decreases in pain during the exercises. These metrics can be tracked to analyze the rehabilitation and management of the patient's recovery. For example, to ensure the patient has the expected joint and muscle function before the conclusion of the therapy.

However, historically, patient adherence to rehabilitation protocols is low. Patients may forget to do the prescribed exercises, they may not complete the full set of exercises, or they may improperly complete the exercises. In addition, they may misrepresent the failure to comply with the protocol to the physiotherapist. This information may be critical for dynamic adjustments to the protocol to ensure a full recovery.

One of the major concerns following an injury and surgical procedure is regaining muscle control. For example, anterior cruciate ligament reconstruction patients often have difficulty performing a static leg raise requiring quadriceps activation. The leg is not able to be held straight in suspension due to rapid atrophy of the muscles and inhibition by pain.

Electromyography (EMG) is the study of human muscle biopotentials, measuring the electrical voltage differential of the muscle. This signal is used to measure the activation signal sent to the muscle to provide muscle force and muscle contraction. Electromyography can give information about the timing and strength of muscle activations through the duration of the physiotherapy. Muscle activation timing and strength information can be useful to ensure the proper function of a muscle through a movement or exercise. In addition, EMG signals may be used for biofeedback to a patient.

Biofeedback is a source of information to the patient about their current activity. Visual biofeedback may provide a patient information about their current exercise or activity so that they may react to the information. One such example is visualizing electromyographic signals which have been shown to be beneficial during the recovery of skeletal injuries. When a patient is unable to perceive muscle activation, visual biofeedback of the muscle contraction aids in the recovery by helping regain control of the muscle. Quickly regaining control of a muscle can speed up the recovery by allowing a patient to correctly perform movements and activities sooner during the rehabilitation. Historically the equipment necessary to facilitate this visual biofeedback has been limited to research labs and medical facilities.

Different treatment and exercise monitoring solutions have been proposed in the art, such as that described in U.S. Pat. No. 8,713,656, entitled “CLOSED-LOOP THERAPY ADJUSTMENT” issued May 20, 2014 to Bourget et al.; U.S. Patent Application Publication number US 2012/0259652, entitled “SYSTEMS AND METHODS FOR REMOTED MONITORING, MANAGEMENT AND OPTIMIZATION PF PHYSICAL TREATMENT” published Oct. 11, 2012 to Mallon et al.; U.S. Patent Application Publication number US 2104/0135593, entitled “WEARABLE ARCHITECTURE AND METHODS FOR PERFORMANCE MONITORING, ANALYSIS AND FEEDBACK” published May 15, 2014 to Jayalth et al.; and U.S. Patent Application Publication number US 2014/0142459, entitled “WEARABLE PERFORMANCE MONITORING, ANALYSIS AND FEEDBACK SYSTEMS AND METHODS” published May 22, 2014 to Jayalth et al. These, however, suffer from various drawbacks and limitations when it comes to monitoring recovery.

This background information is provided to reveal information believed by the applicant to be of possible relevance. No admission is necessarily intended, nor should be construed, that any of the preceding information constitutes prior art or forms part of the general common knowledge in the relevant art.

### SUMMARY

The following presents a simplified summary of the general inventive concept(s) described herein to provide a basic understanding of some aspects of the disclosure. This summary is not an extensive overview of the disclosure. It is not intended to restrict key or critical elements of the disclosure or to delineate the scope of the disclosure beyond that which is explicitly or implicitly described by the following description and claims.

A need exists for a system, device and method for monitoring physical recovery that overcome some of the drawbacks of known techniques, or at least, provides a useful alternative thereto. Some aspects of this disclosure provide examples of such systems, methods and devices, for instance, that allow for the effective monitoring of pain, and the evolution thereof, during such recovery, for example, within the context of a rehabilitation or recovery training program that may involve a series of prescribed exercise routines and the like.

In accordance with one aspect, there is provided a monitoring system for monitoring a physical recovery, the system comprising: a wearable structure to be worn in an area related to the physical recovery; a user movement-related sensor operatively mounted on said wearable structure and operable to monitor a user movement associated with said physical recovery and generate a user movement-related signal representative thereof over time; a user input interface operable to generate a digital pain indicator during said user movement to correlate said digital pain indicator with said user movement; and a data storage device for storing data related to said digital pain indicator against at least a portion of said user movement-related signal to be correlated in monitoring the physical recovery associated with said user movement.

In one embodiment, the wearable structure comprises at least one of a wearable brace, a wearable harness and a wearable compression sleeve.

In one embodiment, the movement-related sensor comprises a kinematic sensor operable to monitor user kinematics associated with said user movement and generate a kinematic signal representative thereof.

In one embodiment, the area is a joint area and wherein said kinematic sensor is operable to monitor a joint angle over time so to correlate a timing of said digital pain indicator with a particular joint angle.

In one embodiment, the pain indicator is a pain onset indicator to be correlated with said particular joint angle as indicating a joint pain onset angle.

In one embodiment, the pain indicator is a pain level indicator to be correlated with said particular joint angle as indicating a joint pain level for said particular joint angle.

In one embodiment, the user movement corresponds with a prescribed exercise and wherein said kinematic sensor is operable to monitor said user movement during performance of said prescribed exercise to correlate a timing of said digital pain indicator with a particular point during performance of said prescribed exercise represented by at least one of a particular user body or body part position, orientation, displacement, velocity and acceleration.

In one embodiment, the pain indicator is a pain onset indicator to be correlated as indicating a pain onset point within said prescribed exercise.

In one embodiment, the pain indicator is a pain level indicator to be correlated as indicating a pain level for said particular point within said prescribed exercise.

In one embodiment, the movement-related sensor further comprises a physiological sensor operable to generate a physiological signal over time to be correlated with said kinematic signal and said digital pain indicator.

In one embodiment, the physiological signal is representative of muscle activation during performance of said prescribed exercise.

In one embodiment, the physiological signal is an electromyography (EMG) signal.

In one embodiment, the movement-related sensor comprises a physiological sensor operable to generate a physiological signal over time to be correlated with said digital pain indicator.

In one embodiment, the user movement corresponds with a prescribed exercise and wherein said physiological sensor is operable to monitor said user movement during performance of said prescribed exercise to correlate a timing of said digital pain indicator with corresponding physiological data during performance of said prescribed exercise.

In one embodiment, the physiological data comprises muscle activation data representative of muscle activation during performance of said prescribed exercise.

In one embodiment, the pain indicator is a pain onset indicator to be correlated as indicating a pain onset point within said prescribed exercise.

In one embodiment, the pain indicator is a pain level indicator to be correlated as indicating a pain level point within said prescribed exercise.

In one embodiment, the data storage device is operable to locally or remotely store data related to said user movement-related signal during a designated period around a timing of said digital pain indicator.

In one embodiment, the user input interface is operatively mounted on said wearable structure.

In one embodiment, the system further comprises an external device having a data processor operable to receive as input said user movement-related signal and said digital pain indicator to automatically correlate a timing of said indicator with said user movement.

In one embodiment, the system further comprises an external device operating said digital data processor, and a communication interface operable between said sensor and said external device to communicate said user movement signal to said external device.

In one embodiment, the user input interface is implemented by said external device.

In one embodiment, the external device is one of a mobile device, a tablet and a computing device, and wherein said user input interface is a graphically rendered input interface implemented by a software application executed by said external device.

In one embodiment, the user movement is associated with a prescribed exercise to be repeated over a time period, and wherein evolution of a value associated with said pain indicator over said time period is correlated with a recovery effectiveness indicator for said prescribed exercise.

In accordance with another aspect, there is provided a monitoring device for monitoring a physical recovery, the device comprising: a wearable structure to be worn by a user in an area related to the physical recovery; a user movement-related sensor operatively mounted on said wearable structure and operable to monitor a user movement associated with the physical recovery and generate a user movement-related signal representative thereof over time; a user input interface operatively mounted on said wearable structure and operable to generate a digital pain indicator during said user movement to correlate said digital pain indicator with said user movement; and a digital output to output data related to at least a portion of said user movement-related signal and to said digital pain indicator to a data storage device to be correlated in monitoring the physical recovery associated with said user movement.

In one embodiment, the device further comprises said data storage device.

In one embodiment, the device further comprises a communication interface for communicating said data to an external device in real-time for processing.

In one embodiment, the wearable structure comprises at least one of a wearable brace, a wearable harness and a wearable compression sleeve.

In one embodiment, the movement-related sensor comprises a kinematic sensor operable to monitor user kinematics associated with said user movement and generate a kinematic signal representative thereof.

In one embodiment, the user movement corresponds with a prescribed exercise and wherein said kinematic sensor is operable to monitor said user movement during performance of said prescribed exercise to correlate a timing of said digital pain indicator with a particular point during performance of said prescribed exercise represented by at least one of a particular user body or body part position, orientation, displacement, velocity and acceleration.

In one embodiment, the pain indicator is a pain onset indicator to be correlated as indicating a pain onset point within said prescribed exercise.

In one embodiment, the pain indicator is a pain level indicator to be correlated as indicating a pain level for said particular point within said prescribed exercise.

In one embodiment, the movement-related sensor further comprises a physiological sensor operable to generate a physiological signal over time to be correlated with said kinematic signal and said digital pain indicator.

In one embodiment, the movement-related sensor comprises a physiological sensor operable to generate a physiological signal over time to be correlated with said digital pain indicator.

In one embodiment, the user movement corresponds with a prescribed exercise and wherein said physiological sensor is operable to monitor said user movement during performance of said prescribed exercise to correlate a timing of said digital pain indicator with corresponding physiological data during performance of said prescribed exercise.

In one embodiment, the physiological data comprises muscle activation data representative of muscle activation during performance of said prescribed exercise.

In one embodiment, the pain indicator is a pain onset indicator to be correlated as indicating a pain onset point within said prescribed exercise.

In one embodiment, the pain indicator is a pain level indicator to be correlated as indicating a pain level point within said prescribed exercise.

In one embodiment, the user movement is associated with a prescribed exercise to be repeated over a time period, and wherein evolution of a value associated with said pain indicator over said time period is correlated with a recovery effectiveness indicator for said prescribed exercise.

In accordance with another aspect, there is provided a computer-implemented method for monitoring a physical recovery through repeated performance of a prescribed user movement over time, the method comprising: receiving as input, for each performance of the prescribed user movement: a user movement-related signal, output by a corresponding user movement-related sensor, representative of the prescribed user movement over time; and a digital pain indicator input via a user input interface during performance of the prescribed user movement; automatically correlating, for each said performance, said digital pain indicator with at least a portion of said user movement-related signal; and digitally tracking physical recovery based at least in part on an evolution of said digital pain indicator correlation over time.

In one embodiment, the user movement-related signal is representative of a recovering joint angle, and wherein a timing of said digital pain indicator is correlated with a joint pain onset angle.

In one embodiment, the portion of said user movement-related signal corresponds with a designated time period around an input timing associated with said digital pain indicator.

In one embodiment, the movement-related sensor comprises a kinematic sensor operable to monitor said user movement to correlate a timing of said digital pain indicator with a particular point during performance of said movement represented by at least one of a particular user body or body part position, orientation, displacement, velocity and acceleration.

In one embodiment, the pain indicator is a pain onset indicator to be correlated as indicating a pain onset point within said prescribed exercise.

In one embodiment, the pain indicator is a pain level indicator to be correlated as indicating a pain level for said particular point within said prescribed exercise.

In one embodiment, the movement-related sensor further comprises a physiological sensor operable to generate a physiological signal over time to be correlated with said kinematic signal and said digital pain indicator.

In one embodiment, the movement-related sensor comprises a physiological sensor operable to generate a physiological signal over time to be correlated with said digital pain indicator.

In one embodiment, the user movement corresponds with a prescribed exercise and wherein said physiological sensor is operable to monitor said user movement during performance of said prescribed exercise to correlate a timing of said digital pain indicator with corresponding physiological data during performance of said prescribed exercise.

In one embodiment, the physiological data comprises muscle activation data representative of muscle activation during performance of said prescribed exercise.

In one embodiment, the pain indicator is a pain onset indicator to be correlated as indicating a pain onset point within said prescribed exercise.

In one embodiment, the pain indicator is a pain level indicator to be correlated as indicating a pain level point within said prescribed exercise.

In one embodiment, the user movement is associated with a prescribed exercise to be repeated over a time period, the method further comprising correlating said evolution with a recovery effectiveness indicator for said prescribed exercise.

Other aspects, features and/or advantages will become more apparent upon reading of the following non-restrictive description of specific embodiments thereof, given by way of example only with reference to the accompanying drawings.

### BRIEF DESCRIPTION OF THE FIGURES

Several embodiments of the present disclosure will be provided, by way of examples only, with reference to the appended drawings, wherein:

FIG. 1 is a schematic diagram of a system for monitoring physical recovery, in accordance with one embodiment;

FIG. 1A is a schematic diagram of the system of FIG. 1 as applied to a knee brace or compression sleeve implementation with communicative relay to a mobile device in accordance with one embodiment;

FIG. 2 is a graphical representation of knee angle data signals generated by a corresponding kinematic sensor in a system for monitoring physical recovery, in accordance with one embodiment;

FIG. 3 is a graphical representation of eletromyographic quadriceps muscle activation data produced from a movement-related signal generated by a corresponding physiological sensor in a system for monitoring physical recovery, in accordance with one embodiment; and

FIG. 4 is flow diagram for an recovery process, in accordance with one embodiment; and

FIGS. 5A and 5B are schematic diagrams of an prescribed exercise executed from respective (standing and lying) starting positions that can be tracked using a physical recovery monitoring system as described herein.

### DETAILED DESCRIPTION

The systems, devices and methods described herein provide, in accordance with different embodiments, different examples in which a physical recovery can be tracked and monitored, for instance, by digitally monitoring and correlating the onset or relative pain experienced during the performance of prescribed exercises associated with this recovery, and how the experience of pain evolves over time and/or throughout a prescribed rehabilitation treatment regime.

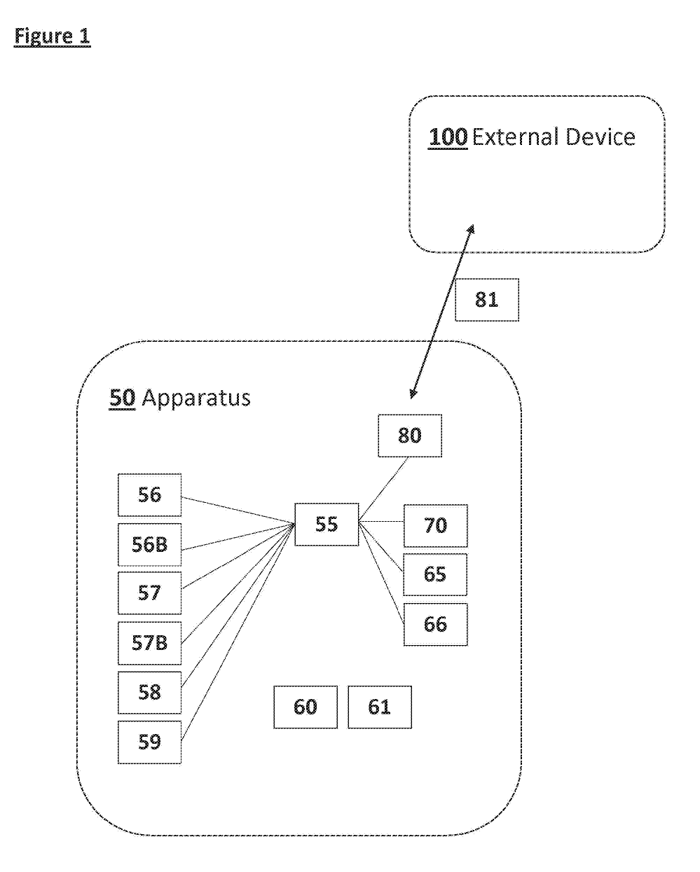

With reference to FIG. 1, and in accordance with one embodiment, a system for monitoring recovery will now be described in general terms. As noted above, in accordance with some embodiments, the systems and devices described herein may provide for effective tracking and assistive feedback in the recovery process from different physical (e.g. joint) injuries, disease and the like.

In the illustrated embodiment of FIG. 1, the system generally includes a wearable device or apparatus 50 to be worn by the user in a physical recover area of interest, and comprising one or more movement-related sensors (e.g. sensors 56, 56B, 57, 57B, 58, 58B and 59) operable to monitor a user movement related to the recovery, such as monitoring the performance of prescribed treatment or rehabilitation exercises, stretches and/or poses, and rest periods therebetween. Movement-related signals/data produced by or from these sensors can then be tracked and correlated, in some embodiments, with one or more input digital pain indicators, for instance input by the user via a corresponding user interface such as pain onset/level button 66 to correspond with movement-related pain, to monitor the user's condition and recovery over time. These and other features will be discussed in greater detail below with reference to these examples.

In the illustrated embodiment of FIG. 1, the wearable apparatus 50 can be placed in a configuration which allows for the various movement-related sensors 56, 56B, 57, 57B, 58, 58B and 59 to be housed in locations surrounding an injured or diseased areas, such as joint, muscle or muscle group, ligaments, tends, and the like. Namely, and as will be appreciated by the skilled artisan, while the following example focuses on the monitoring of recovery related to an injured or diseased joint, physical recovery for other anatomical regions/areas, such as the recovery of different muscles, ligaments and/or tendons, may also or alternatively be monitored using similar equipment. Accordingly, the wearable apparatus 50 may be manufactured of different materials depending on the application at hand, which materials may include, but are not limited to, fabric, jersey and elastic, as can other manufacturing materials and components be used to enhance the wearability of the apparatus 50, such as straps, harnesses, elastics, bands, clips, fasteners, and the like.

With added reference to FIG. 1A, and in accordance with one non-limiting embodiment for monitoring joint recovery, the apparatus 50 may consist of a compression sleeve or brace built for the knee or elbow joints. In another example, the apparatus 50 may be built as a pair of shorts to observe the function of a hip joint, or a shirt to observe the function of a shoulder joint, for example. The apparatus 50 may be tight in specific areas, and conform to the skin to allow for various sensors to make constant contact with the skin of the user, but the entire apparatus 50 need not be tight and conforming. The apparatus 50 may include a rigid structure such as rods, pins or plates if needed to assist housing and placement of the various sensors. In one embodiment the apparatus 50 may be in the form of a joint brace. Other wearable structures may also be considered, such as a brace, a harness, a compression sleeve, a fitted article of clothing such as a glove, sock, shorts, shirt or the like. These, and other examples, will be appreciated by the skilled artisan to fall within the scope of the present disclosure.

The apparatus 50 may be attached to the body by the use of various techniques. In one example, a number of straps may be used by the user to tighten the apparatus/brace/sleeve onto the area (e.g. join, muscle) of interest to be monitored. These straps would allow a patient with limited range of motion to place the apparatus 50 on an injured knee without the need to place the apparatus 50 over the foot, for example. In another embodiment, the apparatus 50 may be designed in a pair of exercise shorts for the rehabilitation of a hip injury. The apparatus 50 may be pulled taught against the skin by a drawstring around the waist to prevent the apparatus 50 from moving out of place. Other such examples of wearable structures amenable for monitoring different anatomical regions should be readily apparent to the skilled artisan.

As noted above, various movement-related sensors may be included to monitor user movements related to the recovery in observing improvements both during the performance of a given movement (i.e. exercise) and throughout the course of a given prescribed exercise regime, for example. In the illustrated embodiments, kinematic sensors (e.g. sensors 56 and 56B) and/or physiological sensors (e.g. sensors 57, 57B and 58) can be mounted in various configurations onto or within the wearable device 50 to detect and monitor different movement-related signals (e.g. kinematic and/or physiological signals) in tracking physical recovery. These sensors may be mounted using any number of different techniques such as sewn, glued, hook and loop, and any other technique that may attach a sensor to the apparatus 50 in a desired or preferred operational configuration and/or position. These sensors 56, 57, 58 may also be mounted in such a way as to be comfortable to the user when worn.

In some embodiments, one or more kinematic sensors 56, 56B may be included to monitor operable to monitor user kinematics associated with a prescribed movement and generate a kinematic signal representative thereof. For example, a kinematic sensor may be used to track a particular user body or body part position, orientation, displacement, velocity and acceleration. For instance, when monitoring a physical joint recovery, one or more kinematic sensors may be used to detect and monitor a joint movement, absolute and/or relative position, and/or angle during performance of a given movement.

Kinematic sensors may include, but are not limited to, accelerometers, gyroscopes, magnetometers, stretch sensors, flex sensors, optical sensors, and potentiometers, among others. Generally, each sensor will be positioned upon wearing the apparatus so to measure the movement or relative movement of a designated body segment. One example sensor setup involves mounting an accelerometer on the thigh and another on the shank in order to measure the relative position between the two segments. This allows for the measurement of knee angle throughout the movements performed by the user. While FIG. 1 depicts two sensors 56 and 56B for the use of movement and angle detection, different embodiments may include more or less sensors depending on the intended application.

In some embodiments, the kinematic sensors(s) may be configured to monitor not only relative anatomical positions and/or angles (e.g. join angle and/or position), but also absolute positions, for example, relative to gravity. This may be particularly helpful when seeking to monitor the proper execution of prescribed exercises, for example. For example, the kinematic sensors 56 may include at least one accelerometer and/or inclinometer to detect a relative orientation of the apparatus 50 relative to gravity, thus allowing for overall spatial recognition to the device beyond joint angles and relative body positions/orientations.

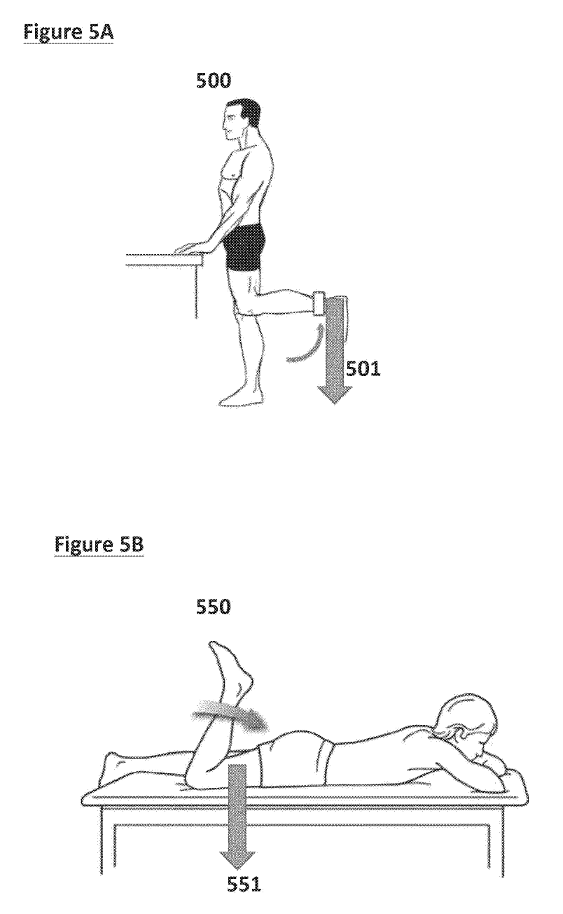

The ability to detect the direction of gravity relative to the user can give useful information to the user, physiotherapist, surgeon or other medical practitioners. One potential use for the gravity vector is to detect if the patient has started an exercise or movement in the correct position. This gives additional information beyond basic the joint angles and/or relative body positions. An example of using the gravity vector to detect exercise position is given in FIG. 5.

FIG. 5 outlines an example exercise: the standing knee flexion. This prescribed exercise calls for the user to be standing and flex their knee behind them while keeping an upright posture relative to a gravity vector 501, as shown at 500. An example of this exercise performed incorrectly is shown at 550, where the user is lying prone on a table perpendicular to the gravity vector 510. Upon using at least one kinematic sensor able to detect user movement relative the direction of gravity while also monitoring a relative knee angle, the device can effectively differentiate between the two examples and thus positively identify when a user preforms the prescribed exercise properly in the proper position.

Accordingly, by including at least one accelerometer or like kinematic sensor, the system may positively distinguish between proper and improper exercise executions, which information can be sent along with other movement-related data to the external device 100 for processing and consumption.

This embodiment of sensors is not limited to only detecting the correct start position of the user but also, if the user has moved from the correct start position during performance of the exercise. For instance, the device can track if the user started the standing knee flexion exercise 500 correctly, but later transitioned to laying on their stomach as shown in 550, or less dramatically, if the user's general posture (e.g. lean forward or backward relative to vertical) changed over time. Other postural variations including, but not limited to, rolling, sliding, translating, leaning, pivoting or the like may also be tracked and used to provided feedback and insight into recovery.

The device 50 may further or alternatively include one or more physiological sensors operable to generate a physiological signal (e.g. muscle activation signal) representative of the user movement. For example, device 50 may include a number of bio potential circuits encompassing one or more electromyographs (EMG), electrocardiographs and/or breath sensors, to name a few. In the illustrated example, EMG circuits 57, 57B may be placed on the apparatus 50 so to collect electromyography data on the muscles surrounding the joint or injury site of interest. For instance, when relating to the human knee, the apparatus 50 may be configured to collect EMG signals on the musculature surrounding the knee joint, such as, but not limited to, vatus lateralis, vatus medialis, vatus intermedius, lateral gastrocnemius, medial gastrocnemius, bicep femoris long head, bicep femoris short head and anterior tibialis. Again, while FIG. 1 depicts only two physiological sensors 57, 57B, in this example for the use of electromyography detection, other embodiments may readily include less than or more than two physiological sensors.

Again with reference to FIGS. 1 and 1A, the apparatus 50 may further comprise a central processing station 55 and/or connected external device 100, which may be configured or operated to collected signals/data from any number of the provided sensors depending on the application at hand, such that multiple sensors and sensor configurations may be provided to accommodate different recovery and/or treatment cycles/regimens employing a same system/device. For instance, use of only a subset of the electromyographic sites available 57, 57B may be pre-decided upon depending on the activity at hand, clinician interest, or other reasons. To give an example, during the “static leg raise exercise”, a physiotherapist may only be interested in the quadriceps muscle group activity and not the hamstring activity to give visual biofeedback to the patient. In another use case, the hamstrings and the quadriceps muscle electromyographic signals from sensors 57, 57B will both be collected during walking to compare against a known activation pattern common between humans for walking. Incorrect activation patterns may allow a physiotherapist to diagnose issues with the rehabilitation.

The electromyography sensors 57, 57B may have a number of different parts and embodiments that allow for the collection of the signal. In one typical embodiment, two electrodes will be positioned to make contact with the skin superficially to the muscle of interest. The two electrodes will be placed proximally and distally along the muscle. These electrodes can thus collect the muscle activation signal as it travels along the muscle. Voltage differences between the muscle activation signals are amplified to get an electromyographic signal. A ground electrode 58 can also be used to get a reference signal for the body away from the EMG collection site. In some cases the circuitry needed to amplify the signals are close to the electrodes themselves and are referred to as “active electrodes”.

Electrodes used to collect electromyographic signals at the electromyographic sensors 57, 57B can be of a number of different electrode styles. A traditional electrode style is called a gel or wet electrode. These electrodes often have a sticky adhesion substrate at the base of the electrode and are often disposable. This substance sticks to the user's skin allowing for continuous and non-slipping contact. Another style of electrodes is called dry electrodes, usually made from a silver or silver-chloride material. These electrodes do not normally use a sticky adhesion to the skin and thus do not require consumables. To ensure continuous signal generation, contact with the skin must be maintained. One possible way to maintain contact with the skin is by applying pressure to the electrode. If the electrodes 57, 57B are placed between the apparatus 50 and the skin, the apparatus 50 may be made tight, causing direct contact between the electrode and the skin using straps or stretchy materials.

The apparatus 50 may include a number of other sensors 59 beyond kinetic sensors 56, 56B and electromyographic sensors 57, 57B, 58. For example, other bio potential circuits may include electrocardiogram, electrocorticogram, or electroencephalogram. An electrocardiogram may be used to track the heart rate of a user during an activity to help track the impact of the movement on the cardiovascular system. Other types of sensors that may be included in the apparatus 50 are barometric, breath, humidity and temperature sensors among others.

As noted above, the apparatus 50 may include a central processing station 55 to couple with the various sensors 56, 56B, 57, 57B, 58, 59. The role of the central processing station 55 may be to collect information on the sensors embodied in the apparatus 50, extract the required information and send the information via the communication a device 80 to an external device 100, for example. The central processing station 55 may be a microcontroller, microprocessor, computer, field programmable gate array, or another processing station capable of collection and manipulating the signals collected from the sensors 56, 56B, 57, 57B, 58, 59.

To connect and/or interconnect the various sensors 56, 56B, 57, 57B, 58, 59 with the processing station 55, a number of wired or wireless connections 60 may be required. Wired connections may allow for either or both communication and power. It is common for sensors 56, 56B, 57, 57B, 58, 59 to need three or more wires for every sensor, positive voltage supply, ground, and a signal cable. In some cases a negative supply cable or a clock cable is also needed. The wired connections 60 are not limited to those mentioned, but rather may include any connection hardware required to provide the functional use of the desired sensors 56, 56B, 57, 57B, 58, 59.

In the illustrated embodiment, the apparatus 50 further comprises a power source 70, such as a reloadable and/or rechargeable battery, to power the apparatus 50 without the inconvenience of a powering wire harness, though wired embodiments may also be contemplated. The power source 70 is also used to power the various sensors on the apparatus 50, processing station, and wireless communication device 80 and any other component that may require power. Power may come from a number of different sources such as a battery, wall plug, or Universal Serial Bus adaptor. It is not necessary for all pieces of the apparatus 50 to be powered from the same power source 70. For instance, each individual electromyographic electrode may be powered by a small wristwatch battery while the processing station may be powered through a USB connection. Not all of the components requiring power must be powered at the same time. In one example, the kinematic (e.g. motion and angle) sensors 56, 56B may be turned off during periods of time where the information is not required to conserve power from the power source 70.

As noted above, the illustrated embodiment also optionally includes a communication device or interface 80 to send the collected information 81 from the processing station 55 to an external device 100. A communication device 80 may include, but is not limited to, a Bluetooth, Zigbee, wifi, or wired connection, amongst other technology readily known in the art for sending digital information 81 between devices. One embodiment of the apparatus 50 includes a Bluetooth communication device to transfer information from the central processing station to a cell phone or other mobile device, for example. The information sent may be information from the whole or subset of the sensor array embedded in the apparatus 50. To give an example, in an apparatus 50 designed for the knee, the knee angle collected from angle measurement sensors 56, 56B during a prescribed exercise may be collected by the central processing station 55 and sent to a smart phone through the communication device 80.

In the illustrated embodiment, information 81 sent from the communication device 80 is sent to an external device 100. An external device may be a cell phone, tablet, computer, hard drive or any other device capable of receiving digital 81. The external device 100 may be used by the user of the device, physiotherapist, clinician or other persons for the use of monitoring and tracking recovery, for example, through the performance of physiotherapy or other physical treatment exercise routine. In other embodiments, the device 50 may also or alternatively be operated as a standalone device, whereby external data communication is not required or available. In such embodiments, the device 500 would generally include an onboard user interface such as a small digital screen or the like to allow for data consumption and user feedback.

In some embodiments, two or more apparatus 50 may be connected together to form a combined apparatus working to gather a larger set of data. These combined devices may be connected using any of the communication devices 80 noted above to communicate with each other and/or with the external device 100. In one scenario, a first apparatus 50, for example attached to the knee in the form of a compression sleeve, and a second apparatus 50 comprised of a pair of shorts for monitoring the hip, may be connected together by a wired connection 80. This wired connect 80 may allow for the transfer of information between devices, such as electromyographic signals, joint angles or other information from other sensors 56, 56B, 57, 57B, 58 and 59. This would allow for a communication, and comparison of such signals between two devices. In the current embodiment, only one communication device 80 would be required to send information 81 to the external device 100 because the information is being shared between the two apparatus. This may be useful to reduce the number of communication channels the external device 100 would require.

In a similar embodiment, an apparatus 50, placed at the ankle and a second apparatus 50 worn on the shoulder may be used without the communication 80 between the two apparatus. It may be advantageous to have both apparatus communicate independently through blue tooth communication devices 80 to the external device 100 as wiring between the two apparatus in this scenario may be cumbersome to the user. To use both devices at a single time, the external device 100 may be configured to communicate with both devices simultaneously.

Surface electromyographic signals measured from the example EMG sensors 57, 57B may be measured up to the range of millivolts depending on the collection site and the strength of the muscle activation, and may be amplified in some embodiments prior to being measured by the onboard processing station 55. Amplification may be performed, for example, by an instrumentation amplifier, given their relatively low noise, and high common-mode rejection ratios. A high pass filter can also be provided to remove unwanted direct current offsets that may have occurred during the amplification process.

Other filters, such as low pass and/or high pass filters, may also be considered for example, to remove unwanted attributes from the signal. For instance, high-pass filtering may be used in situations where the signal is subject to a DC offset bias. This may be used if the average value of the signal is not zero. This may hinder the use of EMG signals for other analysis such as envelope filtering and muscle activation threshold detection, which require the average signal to be equal or close to zero, as will be readily understood by the person of ordinary skill in the art using standard EMG analysis techniques.

Additional or alternative signal filters may also be included, for example, to filter kinematic signals such as that provided by angle sensors or the like due to the inherent nature of noise in a number of these sensor embodiments, namely to remove high frequency noise from the signals.

In one particular example, analog circuitry may be included to apply, low and high pass filters, among others. Analog high and low pass filters often include operational amplifiers, resistors and capacitors among others. In another example, digital filtering may accomplished by central processing station 55 and/or on the external device 100. For example, digital filtering may be accomplished by sampling the sensor data in uniform time intervals and applying a known filtering algorithm to the data.

With reference still to FIG. 1, the apparatus 50 may further include a built in data storage unit 65 or the like. This data storage unit may be used to allow for onboard data storage and thus reduced data communication requirements between the apparatus 50 and the external device 100. In one example operating a Bluetooth communication device 80, the user may find that they are not able to communicate with the external device 100 because they have moved out of range of the device. The built in data storage unit 65 would thus be able to save all information regarding the activities they are performing, and transfer the information to the external device 100 at a later time, when they are back in range. This feature may be used for example if a patient is required to go for a walk and does not wish to bring the external device along with them.

In another example the built in data storage unit 65 may be used if the user does not own or have access to an external device 100 but wishes to still be monitored with the apparatus 50. The data could then be transferred at a later date to an external device when one is available.

The built in data storage unit 65 may be implemented using any number of storage devices and protocols such as but not limited to, random access memory, solid state drive, hard drives, or EEPROM. Any device capable of storing digital data may be used.

As noted above, sensor data may be processed at various points in the apparatus 50 and/or through the external device 100. For instance, data processing can start at the site of the sensor through the use of analog filtering, but can occur as late as the external device 100. This allows for data to be processed in an efficient manner because the process can be started early, at the site, or late on the external device depending on the needs of the user. If the apparatus encompasses many sensors, it may produce too much raw data for the central processing station 55 or the external device 100 to process the quantity of data in a timely fashion. Using analog filters, or starting the data processing at the site of the data collection, or at the central processing station allows for the processing to be completed sequentially at a number of different places on the device. This can help reduce the speed necessary in any one location, such as at the site, central processing station 55 or the external device 100, thus allowing for more complex analysis and data processing to take place overall.

In one example, a high pass filter is used on the electromyography data to remove a DC offset in the signal, using an analog filtering technique at the site of the signal generation. This signal is then processed in the central processing station using a rectifier and low pass filter to get the signal envelope of the data, a common technique used in EMG analysis. This data is then transported to the external device 100 over the communication device 80. The external device may use this enveloped data to determine if the muscle has been activated using a threshold technique and a statistical model. The process of this muscle activation analysis is shared across the site of the measurement, the central processing station 55 and the external device 100, reducing the amount of processing power required at the external device 100 if the raw signal was otherwise being transported. These techniques become increasingly important with increasing sensor demands.

Continuous signal measurement and storage can lead to large quantities of unwanted data being stored. This can lead to exceeding the data storage capabilities of the apparatus 50 or the external device 100 is a short period of time. Therefore it may be important for only desired data to be stored for future use. In the example noted above relating to the monitoring and storage of knee angle data for a given exercise, a continuous stream of unprocessed or processed data may be available from the angle sensors for storage and future use. However, the user of the current device may wish to only record the maximum knee angle achieved within every 5-second period, namely the period of a recommended exercise from their physiotherapist. If only this maximum knee angle is stored, the amount of data storage required by the device is thus significantly reduced, allowing for data to be collected on many more occasions before exceeding the capacity of the storage unit. Likewise, physiological signals related to this exercises may also only be of particular interest in segments at and around this maximum knee angle, thus further reducing the amount of physiological data required to time segments correlated to these segments of interest. These and other such examples should be readily apparent to fall within the general scope and nature of the present disclosure.

Still with reference to FIG. 1, the apparatus 50 may further include a modality holder 61, for applying medical modalities common in medical and physiotherapy applications. These modalities may include, but not limited to, ice packs, heating packs or electrical stimulation. The modality holder may encompass different modality attachment means, such as a pouch, hook and loop, button, tape, snaps or other. Modalities are commonly used as a physical treatment to an injury. For instance, ice packs are commonly used to reduce swelling at the injury site, while heat packs are used to increase blood flow to the site.

As introduced above, one metric relied upon during a rehabilitation process to monitor recovery is the pain experienced by the patient when static and/or during performance of different movements and/or exercises. For example, it is often a primary goal of the rehabilitation to reduce the pain while simultaneously increasing the amount of physical activity and range of motion of the recovering joint or body area. As pain is not readily quantifiable through signals captured by the kinematic or physiological sensors noted above, the apparatus 50 and/or external device 100 will generally further include a user input interface 66 to simultaneously track pain perceived by the user of the device, either during performance of a given treatment or rehabilitation exercise, and/or after such performance over time.

For example, in one embodiment, an integrated or communicatively linked pain button 66 may be included. Accordingly, if the user experiences pain while wearing the apparatus 50, they can be advised to press the pain button. This will allow the data processing to include pain, as a variable to be tracked simultaneously with other metrics, such a kinematic and/or physiologic data. For example, the user interface 66 may be operated to generate a digital pain indicator upon activation by the user so to correlate a pain onset and/or level against corresponding kinematic and/or physiological data. Such digital pain indicators may be stored and tracked via the apparatus 50 and/or external device 100 to provide feedback on the user's recovery through corresponding exercises and/or help locate the source or contributing factors to persisting pain or discomfort (e.g. where pain levels and/or pain free range of motion do not sufficiently improve).

Generally, the pain indicator interface may take various forms, and can be included as a user input interface on the apparatus 50, the external device 100, or among the software provided in the external device 100, among others. When implemented on the device 50, the button 66 may take a number of different forms, which may include, but are not limited to, a slider, dial, potentiometer, pressure sensitive film, and colour changing button, to name a few examples. Other hardware-enabled interfaces may also be considered, as will be readily appreciated by the skilled artisan.

Software and/or graphically-implemented pain indicator interfaces, for example executed on the apparatus 50 and/or external device 100, may also or alternatively include, but are not limited to, different graphically rendered touch-sensitive input interfaces such as a dynamic pain scale slider or dial, colour-coded button, dropdown pain scale menu, discrete selectable scale buttons, checkboxes or radio buttons, and the like. As will be appreciated by the skilled artisan, different hardware, firmware and/or software implemented user interfaces may be considered either on the apparatus 50 and/or external device 100 to provide similar effects, features and functions, as noted above, without departing from the general scope and nature of the present disclosure.

The pain button can have multiple functions of use. In one embodiment, when the user presses the pain button 66, a time period of kinematic and/or physiological data leading up to the pain event is stored for further analysis. For example, 30 seconds of detailed knee angle data may be collected to be analyzed around an indicated pain point or level. This information may be used to detect if a patient has a specific knee angle that is giving them issues, and can be used by a physician, doctor or others for help in diagnosing a problem. The data logged from such an example can be analyzed to store the maximum knee angle from the detailed information. In one example, a patient may experience a sharp pain in a knee joint when they flex their knee to 120 degrees. If the pain button 66 is repeatedly pressed when the patient experiences pain at 120 degrees, the maximum knee angle from the multiple pain events would show a corresponding maximum knee angle at 120 degrees. Such information can give insight into the injury over time, as can it provide information on recovery if and when the pain onset indicator is progressively applied to greater and greater maximum knee angles over time.

This system can, for instance, be helpful to give insight to a physician, doctor or others for extended periods of time over multiple days, weeks or months, where a maximum knee angle at which a patient experiences onset pain may increase over time, potentially indicating recovery. A typical recovery will see the maximum knee angle at which pain occurs increase throughout the recovery process, and may be considered to be a normal occurrence in the recovery process. In another example, the maximum knee angle does not change with the pain button being more or less consistently pressed to correspond with the same maximum angle over time, thus potentially indicating a complication or stalling of the recovery.

In some embodiments, the pain indicator interface 66 may also or rather provide for input as to a pain level or intensity. For example, a pain level or intensity may be indicated by the length of time a corresponding pain button is pressed, or again by a dynamically adjustable pain scale input such as a dial or slide bar. For example, the user may push the button for longer if the pain experienced was greater, or again input a corresponding pain value based on a predefined pain scale and scaling input interface. This added detail may give insight to the user, physiotherapists, doctors, patients among others, and information about the trends in the rehabilitation and/or program.

Furthermore, pain that users experience can often be categorized into movement and non-movement related pain. Movement related pain can be deduced by correlating user pain indicator inputs with movement-related signals such as kinematic and/or physiological (e.g. muscle activation) signals to see what types of movement patterns were being performed at the time the pain was recorded. A number of different scenarios can be identified with a combination of sensors including joint flexion, contraction of the muscles, instability, twisting, falling, velocity, activity intensity and running, for example.

In a similar embodiment, a patient may push the pain button 66 when they are not actively performing a movement or exercise. Pushing the button 66 in this scenario may indicate non-movement related pain. For example, this pain may be caused by muscle spasm, swelling or nerve related injury. By analyzing the activities performed in close time proximity to the pain event can help distinguish between mechanical and non-mechanical induced pain.

In another embodiment, pain tracking may be completed on the external device to track with a questionnaire. The questionnaire may ask the patient to describe their pain on scale from 1-10 at the end of an exercise, exercise set, at the end of a day, or physical activity, among others. For example the questionnaire may ask the patient to rate the pain in their joint during the previous activity from 0-10 with 0 being no pain, and 10 being the most pain imaginable. In-contrast to the pain button where the user would be encouraged to press the button if there is a shot of pain, the pain scale would be able to address the long term pain associated with a given exercise, exercise set, day or other activities. In yet another example, a post-exercise pain level questionnaire may be correlated with in-exercise input pain indicators, for example, to associate a perceived pain level to such input indicators after the fact so not to complicate pain indicator entries during performance of the exercise.

This information, similar to that of the pain button, can give insight to the physiotherapists, doctors, patients among others information about the trends in the rehabilitation and/or program. For example, a decrease in the reported pain for a stretching exercise over multiple sessions may suggest that the joint is healing, and becoming less stiff. In a counter example, if the pain is reported to be increasing while the range of joint motion is decreasing, it could indicate a complication in the healing process. These are examples of potential uses, and not intended to limit the uses of the herein described embodiments.

As noted above, collecting pain information alongside of the data collected through the various sensors 56, 56B, 57, 57B, 58, 59 allows for the recovery analysis to be more insightful and correspond to the healing process beyond the individual capabilities of the individual sensors.

In some embodiments, the pain button 66, pain scale, etc., may be used to help detect if the user should take (more) pain medication. For example, the external application may request for the user to input when they take a pain medication in order to keep track of quantity of dosing. Pain medication often comes with a window where it is acceptable to take more medication before it has fully worn off. If a user is pressing the pain button repeatedly during this window, the application may suggest to the user that they can take more medication at this time.

Although some embodiments have the pain tracking completed through the external device 100, they are still part of the general scope and nature of the present disclosure as the intended use is for recovery, injury and deterioration tracking and rehabilitation among others.

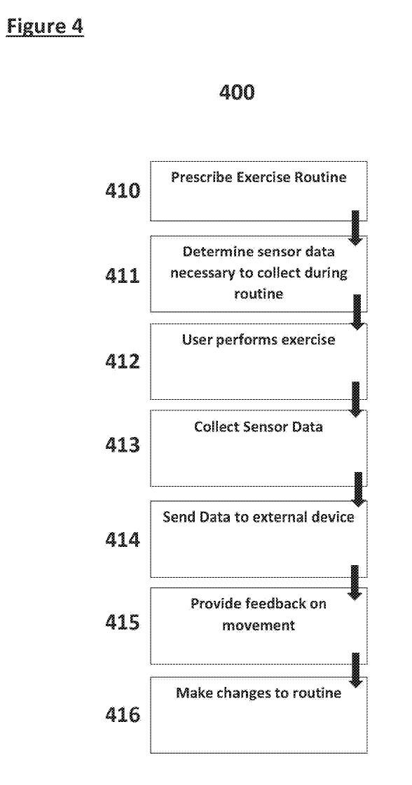

The embodiments of the system and device described herein may used in different ways, as can the data output therefrom be used to provide different feedback and insight into a user's rehabilitation and recovery. As shown for example in FIG. 4, which illustrates an example use of the described system, a physiotherapist may request the user to perform a “knee extension exercise”, for example, as a prescribed exercise routine 410. Using the external device 100, the physiotherapist requests information at 411 from a kinematic knee-angle sensing device 56, 56B. The user performs the exercise at 412 and the sensor data is collected at 413, which is sent at 414 using the wireless communication device 80. The information 81 can be displayed on a mobile device at 415.

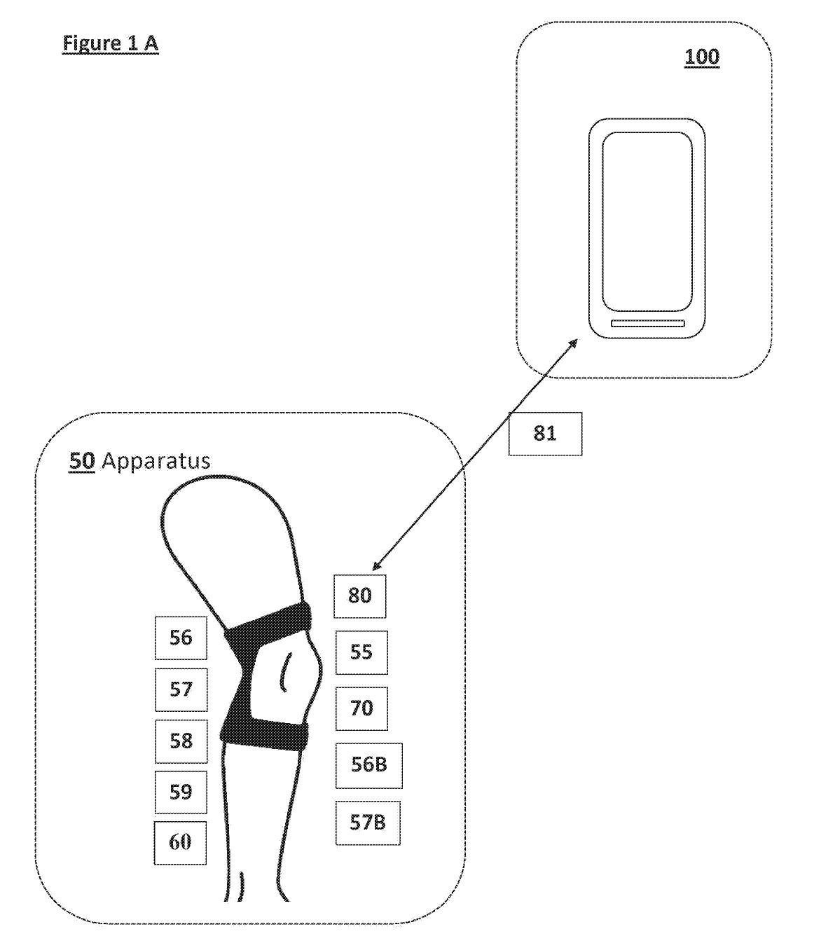

As shown in FIG. 2, feedback data can take the form of a graph 200 of the sensed knee angle data extracted from sensing device 56, 56B for three repetitions from the “knee extension exercise”. The first repetition 210 has the largest maximum knee angle, the second repetition 211 has a smaller maximum knee angle and repetition 212 has the smallest maximum knee angle. This information may indicate to a physiotherapist that the patient is struggling to have a consistent knee angle between repetitions and may lead to a change in rehabilitation protocol at 416 such as a different exercise or the use of visual biofeedback.

For example, the physiotherapist may determine the lack of consistency in knee angle seen in 200 may be due to a lack of feedback from the muscle receptors in the quadriceps muscle group and may prescribe a visual biofeedback exercise during the same knee extension exercise 416, thus restarting process 400 with a new routine 410 encompassing visual biofeedback provided via a new set of sensor data selected at 411 as being provided by a set of complimentary physiological sensors such as electromyography sensors. The user can then again perform the prescribed exercise at 412 and the sensor data is collected at 413, which is again sent at 414 using the wireless communication device 80.

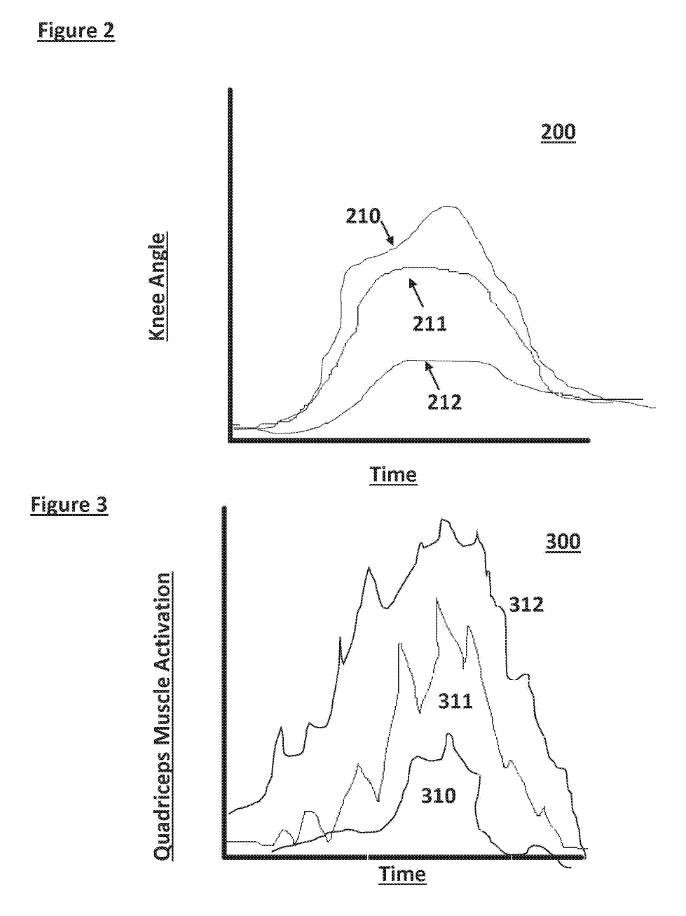

As shown in FIG. 3, complimentary feedback data can now also take the form of physiological data signal graph 300, providing visual feedback as to the quadriceps muscle activation during three repetitions 310, 311, 312. During the first repetition the patient would view the signal 310, receiving this visual biofeedback. This feedback may enable the patient to gain a better sense for their muscle activation and perform a larger activation 311 on the second repetition and an even larger activation during the third repetition 312. Due to the success biofeedback had on increasing the quadriceps activation level, the physiotherapist may decide to include visual biofeedback in subsequent physiotherapy exercise routines 416.

Likewise, pain onset or level data (not shown) may be overlaid or otherwise correlated with kinematic and/or physiological knee angle data to provide further insight as to an appropriateness of the prescribed rehabilitation exercise and/or the recovery progress over time. For example, a pain onset knee angle may be shown to escalate with repetition providing further guidance on diagnosis and treatment options that may not shown up using physiological feedback. Furthermore, tracking pain level and/or onset against such kinematic and/or physiological feedback data over time may be used to confirm or challenge reported or perceived recovery, and encourage further rehabilitation or the pursuit of alternative exercise routines.

While the present disclosure describes various example embodiments, the disclosure is not so limited. To the contrary, the disclosure is intended to cover various modifications and equivalent arrangements included within the general scope of the present disclosure. Various components illustrated in the figures may be implemented as hardware and/or software and/or firmware on a processor, ASIC/FPGA, dedicated hardware, and/or logic circuitry. Also, the features and attributes of the specific embodiments disclosed above may be combined in different ways to form additional embodiments, all of which fall within the scope of the present disclosure. Although the present disclosure provides certain preferred embodiments and applications, other embodiments that are apparent to those of ordinary skill in the art, including embodiments which do not provide all of the features and advantages set forth herein, are also within the scope of this disclosure. Accordingly, the scope of the present disclosure is intended to be defined only by reference to the appended claims.

What is claimed is:

1. A monitoring system for monitoring a physical recovery, the system comprising:
a wearable structure configured to be worn in an area related to the physical recovery;
a user movement-related sensor mounted on said wearable structure, and configured to monitor a user movement associated with said physical recovery and to generate a user movement-related signal representative thereof over time;
a user input interface configured to generate a digital pain indicator during said user movement to correlate said digital pain indicator with said user movement; and
a data storage device configured to store data related to said digital pain indicator against at least a portion of said user movement-related signal to be correlated in monitoring the physical recovery associated with said user movement.

2. The system of claim 1, wherein said user movement-related sensor comprises a kinematic sensor configured to monitor a joint angle over time so as to correlate a timing of said digital pain indicator with a particular joint angle movement and to generate a kinematic signal representative thereof;
wherein said digital pain indicator is a pain onset indicator correlatable with said particular joint angle as indicating a joint pain onset angle; and
wherein said digital pain indicator is a pain level indicator correlatable with said particular joint angle as indicating a joint pain level for said particular joint angle.

3. The system of claim 2, wherein said user movement corresponds with a prescribed exercise and wherein said kinematic sensor is configured to monitor said user movement during performance of said prescribed exercise to correlate a timing of said digital pain indicator with a particular point during performance of said prescribed exercise represented by at least one of: a particular user body or body part position, orientation, displacement, velocity or acceleration.

4. The system of claim 1 wherein said user movement-related sensor further comprises a physiological sensor configured to generate a physiological signal over time to be correlated with a kinematic signal and said digital pain indicator.

5. The system of claim 4, wherein said physiological signal is an electromyography (EMG) signal.

6. The system of claim 1, wherein said data storage device is configured to locally or remotely store data related to said user movement-related signal during a designated period around a timing of said digital pain indicator; and
said user input interface mounted on said wearable structure.

7. The system of claim 1, further comprising an external device including a data processor configured to receive as input said user movement-related signal and said digital pain indicator to automatically correlate a timing of said indicator with said user movement; and
a communication interface configured to communicate with said sensor and said external device to communicate said user movement-related signal to said external device, wherein said user input interface is a graphically rendered input interface implemented by a software application executed by said external device.

8. A computer-implemented method for monitoring a physical recovery through repeated performance of a prescribed user movement over time, the method comprising:
receiving as input, for each performance of the prescribed user movement:
a user movement-related signal, outputted by a corresponding user movement-related sensor, representative of the prescribed user movement over time; and
a digital pain indicator inputted via a user input interface during performance of the prescribed user movement;
automatically correlating, for each said performance, said digital pain indicator with at least a portion of said user movement-related signal; and
digitally tracking physical recovery based at least in part on an evolution of said digital pain indicator correlation over time,
wherein the method is performed by at least one processor.

9. The method of claim 8, wherein said user movement-related signal is representative of a recovering joint angle, and wherein a timing of said digital pain indicator is correlated with a join pain onset angle.

10. The method of claim 8, wherein said portion of said user movement-related signal corresponds with a designated time period around an input timing associated with said digital pain indicator.

11. The method of claim 8, wherein said user movement-related sensor comprises a kinematic sensor configured to monitor said user movement to correlate a timing of said digital pain indicator with a particular point during performance of said user movement represented by at least one of: a particular user body or body part position, orientation, displacement, velocity or acceleration.

12. The method of claim 11, wherein said digital pain indicator is a pain onset indicator to be correlated as indicating a pain onset point within a prescribed exercise.

13. The method of claim 11, wherein said digital pain indicator is a pain level indicator to be correlated as indicating a pain level for said particular point within a prescribed exercise.

14. The method of claim 11, wherein said user movement-related sensor further comprises a physiological sensor configured to generate a physiological signal over time to be correlated with a kinematic signal and said digital pain indicator.

15. The method of claim 8, wherein said user movement-related sensor comprises a physiological sensor configured to generate a physiological signal over time to be correlated with said digital pain indicator.

16. The method of claim 15, wherein said user movement corresponds with a prescribed exercise and wherein said physiological sensor is configured to monitor said user movement during performance of said prescribed exercise to correlate a timing of said digital pain indicator with corresponding physiological data during performance of said prescribed exercise.

17. The method of claim 16, wherein said physiological data comprises muscle activation data representative of muscle activation during performance of said prescribed exercise.

18. The method of claim 15, wherein said digital pain indicator is a pain onset indicator to be correlated as indicating a pain onset point within a prescribed exercise.

19. The method of claim 15, wherein said digital pain indicator is a pain level indicator to be correlated as indicating a pain level point within a prescribed exercise.

20. The method of claim 8, wherein said user movement is associated with the prescribed exercise to be repeated over a time period, the method further comprising correlating said evolution with a recovery effectiveness indicator for said prescribed exercise.
